(19) United States
(12) Patent Application Publication
(10) Pub. No.: US 2023/0374084 A1
(43) Pub. Date: Nov. 23, 2023

(54) POTENT AND SELECTIVE INHIBITORS OF NAV1.7

(71) Applicant: ALDEVRON, LLC, Fargo, ND (US)

(72) Inventors: Slobodan PAESSLER, Fargo, ND (US); Veljko VELJKOVIC, Fargo, ND (US)

(73) Assignee: ALDEVRON, LLC, Fargo, ND (US)

(21) Appl. No.: 18/027,701

(22) PCT Filed: Sep. 22, 2021
(86) PCT No.: PCT/US2021/051550

§ 371 Date: Mar. 22, 2023

Related U.S. Application Data
(60) Provisional application No. 63/082,088, filed on Sep. 23, 2020.

(51) Int. Cl. C07K 14/435

(52) U.S. Cl. CPC C07K 14/435; A61K 38/00

(57) ABSTRACT

The present disclosure relates to a peptide and its analogs that selectively inhibit the Na_{V}1.7 sodium channel. The present disclosure also relates to pharmaceutical compositions useful for treating or preventing a disorder responsive to the blockade of sodium ion channels, especially Na_{V}1.7 sodium ion channels. The present disclosure provides methods of treating a disorder responsive to the blockade of sodium channels, and particularly Na_{V}1.7 sodium channels, in a mammals. The present disclosure further provides compositions and methods for providing analgesia by administering a peptide of the invention.

### CROSS-REFERENCE TO RELATED APPLICATIONS

This application is a PCT International Patent Application and claims priority to U.S. Provisional Patent Application No. 63/082,088, filed Sep. 23, 2020; the disclosure of which is hereby incorporated by reference in its entirety.

### TECHNICAL FIELD

This invention is in the field of peptide chemistry. The present disclosure relates to novel peptides and the use of these peptides as blockers of sodium (Na⁺) channels.

### BACKGROUND

Voltage-gated sodium channels (VGSC) are glycoprotein complexes responsible for initiation and propagation of action potentials in excitable cells such as central and peripheral neurons, cardiac and skeletal muscle myocytes, and neuroendocrine cells. Na_{V} channels are responsible for generating the Na⁺ currents underlying the initiation and propagation of action potentials in nerves and muscle fibers.

Na_{V} channels consist of an α-subunit, which can be coupled to one or two β-subunits. The α-subunit, which forms the core of the channel and is responsible for voltage-dependent gating and ion permeation, are large proteins composed of four homologous domains. Each domain contains six α-helical transmembrane spanning segments. In humans there are nine different a subunits, Na_{V}1.1, Na_{V}1.2, Na_{V}1.3, Na_{V}1.4, Na_{V}1.5, Na_{V}1.6, Na_{V}1.7, Na_{V}1.8, and Na_{V}1.9, encoded by the genes SCN1A, SCN2A, SCN3A, SCN4A, SCN5A, SCN8A, SCN9A, SCN10A, and SCN11A, respectively. See Ruiz et al. J. Med. Chem. 2015, 58, 7093-7118. The high degree of amino acid sequence homology among the different Nav subtypes makes finding subtype-selective ligands extremely difficult.

Na_{V} channel α-subunits show tissue specific expression profiles (Table 1). Na_{V}1.1, Na_{V}1.2, and Na_{V}1.3 subtypes are expressed in the central nervous system (CNS). Na_{V}1.6 is expressed in both the peripheral and central nervous system, whereas Na_{V}1.7, Na_{V}1.8, and Na_{V}1.9 are mostly restricted to the peripheral nervous system (PNS). Na_{V}1.4 and Na_{V}.5 channels are abundant in skeletal and cardiac muscles, respectively. See Goldin, A. L. Ann. N. Y. Acad. Sci. 1999, 868, 38-50. Mutations in α-subunit genes have been linked to paroxysmal disorders such as epilepsy, long QT syndrome, and hyperkalemic periodic paralysis in humans, and motor endplate disease and cerebellar ataxia in mice. See Mulcahy, et al. J. Med. Chem. 2019, 62, 8695-8710.

TABLE 1
Voltage-gated Na⁺ channel gene family

| Type | Gene Symbol | Tissue Distribution | Indication |
|---|---|---|---|
| Nav1.1 | SCN1A | CNS/PNS | Pain, epilepsy, |
| | | | neurodegeneration |
| Nav1.2 | SCN2A | CNS | Pain, epilepsy, |
| | | | neurodegeneration |
| Nav1.3 | SCN3A | CNS | Pain, Epilepsy |
| Nav1.4 | SCN4A | Skeletal muscle | Myotonia |
| Nav1.5 | SCN5A | Heart muscle | Arrhythmia, long |
| | | | QT |
| Nav1.6 | SCN8A | CNS/PNS | Pain, movement |
| | | | disorders |
| Nav1.7 | SCN9A | PNS | Pain |
| Nav1.8 | SCN10A | PNS | Pain |
| Nav1.9 | SCN11A | PNS | Pain |

A number of clinically prescribed small molecule therapeutics modulate Na_{V} activity such as local anesthetics, anticonvulsants, and antiarrhythmics. Goldin, et al. Ann. N. Y. Acad. Sci. 1999, 868, 38-50. Most drugs exhibit limited Na_{V} subtype selectivity and the design of therapeutics targeting Na_{V}s is further complicated by the difficulty of understanding the biophysical and pharmacodynamic consequences of ligand binding to different conformations of the channel. Kingwell, K. Nature Rev. Drug Dis. 2019, 18, 321-323. The finding that certain Na_{V} isoforms, particularly Na_{V}1.7, Na_{V}1.8, and Na_{V}1.9, are expressed predominantly in unmyelinated and small diameter myelinated afferents that transmit nociceptive signals has led to widespread efforts to discover selective inhibitors of these particular subtypes. Cummins et al. Pain 2007, 131(3), 243-257.

Interest in the discovery of selective inhibitors of Na_{V}1.7 has been fueled by the findings that a rare genetic condition in which the channel is nonfunctional leads to insensitivity to pain. Nielson et al. Pain, 2009, 143(1), 155-158. On the basis of the biophysical properties of Na_{V}1.7, localization in small diameter peripheral afferents, human genetic findings, and studies in transgenic mice, Na_{V}1.7 has been vaunted as one of the best validated targets for analgesic drug discovery. Dib-Hajj, et al. Nat. Rev. Neurosci. 2013, 14(1), 49-62. The availability of safe and effective Na_{V}1.7 inhibitors would potentially reduce dependence on opioids for pain treatment. Volkow, et al. N. Engl. J. Med. 2017, 377(4), 391-394. However, selectivity is necessary for drug safety given the obligatory role of off-target Na_{V} isoforms in central and peripheral neuronal conduction (Na_{V}1.1-1.3, Na_{V}1.6), skeletal muscle contraction (Na_{V}1.4), and the cardiac action potential (Na_{V}1.5). Catterall et al. Pharmacol. Rev. 2005 57(4), 397-409.

All Na_{V} subtypes known to date can be classified by their sensitivity to the guanidine-based neurotoxin TTX, a toxin isolated from the puffer fish. Na_{V} 1.1, Na_{V} 1.2, Na_{V}1.3, Na_{V}1.4, Na_{V}1.6, and Na_{V} 1.7 are blocked by low nanomolar concentrations of TTX; therefore, these subtypes are classified as TTX-sensitive, whereas, Na_{V} 1.5, Na_{V} 1.8, and Na_{V} 1.9 are inhibited by only high micromolar TTX concentrations and are considered TTX resistant channels. Na_{V} channels are also known targets for a broad range of natural neurotoxins such as tetrodotoxin (TTX), saxitoxin (STX), and batrachotoxin (BTX) as well as peptide toxins isolated from the venoms of scorpions, spiders, sea anemones, and cone snails. See, Billen et al. Cur. Pharm. Des. 2008 14, 2492-2502.

The polypeptide toxins from the tarantula Thrixopelma pruriens (protoxins) are members of the inhibitory cysteine-knot family of protein toxins, which contain 30 to 35 amino acid residues and three disulfide bridges. Protoxin I (ProTx I) and Protoxin II (ProTX II) are T. pruriens peptide toxins that have three cystine bridges in the connectivity pattern C₂ to C₁₆, C₉ to C₂₁, and C₁₅ to C₂₅. ProTxI and ProTx II inhibit activation of sodium channels, including Na_{V}1.7. (Scmalhofer et al. Molecular Pharm. 2008, 74, 1476-1481. Other peptide inhibitors of VGSCs isolated from spider venoms and peptide analogs were reported in Murray et al., Potent and selective inhibitors of Na_{V}1.3 and Na_{V}1.7, WO 2012/125973 A2; Meir et al., Novel peptides isolated from spider venom and uses thereof, US 2011/0065647 A1; Lampe et al., Analgesic peptides from venom of Grammostola spatulata and use thereof, U.S. Pat. No. 5,877,026; Park et al., Analogs of sodium channel peptide toxin, WO 2012/004664 A2. The production of toxin peptides is a complex process in venomous organisms, and is an even more complex process synthetically. Due to their conserved disulfide structures and need for efficient oxidative refolding, toxin peptides present challenges to synthesis. See, Steiner et al. J. Pept. Sci. 2011 17(1) 1-7; Gongora-Benitez et al. Biopolymers Pept. Sci. 2011, 96(1) 69-80. Although toxin peptides have been used for years as highly selective pharmacological inhibitors of ion channels, the high cost of synthesis and refolding of the toxin peptides and their short half-life in vivo have impeded the pursuit of these peptides as a therapeutic modality. As a result, far more effort has been expended to identify small molecule inhibitors as therapeutic antagonists of ion channels. However, research related to small molecule inhibitors has failed to yield any viable candidates to date. Presumably due to a lack of selectivity among sodium channel subtypes. Thus, there remains a need for more effective and safer analgesics that work by blocking VGSCs.

### SUMMARY

The present disclosure provides compositions of matter, and the pharmaceutically acceptable salts, prodrugs and solvates thereof, which are useful as blockers of sodium (Na⁺) channels, and particularly Na_{V}1.7 channels. In preferred embodiments the composition of matter comprises of a peptide which exhibits selectivity as Na_{V}1.7 channel blocker in the absence of disulfide structures.

In one embodiment, the present invention is directed to a composition of matter comprising of a peptide comprising the amino acid sequence SEQ ID NO 1: SDEIPATFGGGTDAGL (Peptide 1).

In one embodiment, the present invention is directed to a composition of matter comprising of a peptide comprising the amino acid sequence SEQ ID NO 2: DCLGLFRKCIPDMLKCCRFNLVCSRLHKWCKYVF (Peptide 3).

In one embodiment, the present invention is directed to a composition of matter comprising of a peptide comprising the amino acid sequence SEQ ID NO. 3: DCLGLFRKCIPDNDKCCRPNFVCSRTHKVCFYVL (Peptide 4).

In one embodiment, the present invention is directed to a composition of matter comprising of a peptide comprising the amino acid sequence SEQ ID NO. 4: DCLGLFRKCIPDNDKCCRPNLVCSRTHKVCFYVL (Peptide 5).

In one embodiment, the present invention is directed to a composition of matter comprising of a peptide comprising the amino acid sequence SEQ ID NO 5: DCLGMFRKCLPDDDKCCRPNLVCSRTHKWCRLVL (Peptide 6).

In one embodiment, the present invention is directed to a composition of matter comprising of a peptide comprising the amino acid sequence SEQ ID NO 6: IGEKVTIRLITSTNINDDFNIYQQKPGEPPKLL (Peptide 7).

In one embodiment, the present invention is directed to a composition of matter comprising of a peptide comprising the amino acid sequence SEQ ID NO 7: DCLGFMRKCLSTTDLDDDWNCCRPNLVCSRTHKWCKYVF (Peptide 8).

In one embodiment, the present invention is directed to a composition of matter comprising of a peptide comprising the amino acid sequence SEQ ID NO 8: YCQRFMLTCDSKKACCEGLRCKLLCRKII (Peptide 10).

In one embodiment, the present invention is directed to a composition of matter comprising of a peptide comprising the amino acid sequence SEQ ID NO 9:YCQRWLWTCDSKKACCEGLRCKLWCRKII (Peptide 11).

In one embodiment, the present invention is directed to a composition of matter comprising of a peptide comprising the amino acid sequence SEQ ID NO 10: YCLGFMRKCDSERKCCEGMVCRLWCKRRLW (Peptide 12).

In one embodiment, the present invention is directed to a composition of matter comprising of a peptide comprising the amino acid sequence SEQ TD NO 11: SCRRAWMACDTAKVCCNPIKCRVACKRVL (Peptide 13).

The present disclosure further provides pharmaceutical compositions comprising an effective amount of a peptide comprising of an amino acid sequence selected from the group consisting of SEQ ID NOs 1-11, or a pharmaceutically acceptable salt, prodrug or solvate thereof, in a mixture with one or more pharmaceutically acceptable carriers. Pharmaceutical compositions of the present disclosure are useful for treating or preventing a disorder responsive to the blockade of sodium ion channels, especially Na_{V}1.7 sodium ion channels.

The compositions of the invention provide an effective method of treating, or preventing, pain, for example acute, persistent, or chronic pain. Selectivity against off-target sodium channels. In some embodiment the compositions of the invention provide selectivity against those Na_{V} channels governing cardiac excitability (Na_{V}1.5) and skeletal muscle excitability (Na_{V}1.4), is cardinal for any systemically delivered therapeutic. In other embodiments compositions of the present invention provide such selectivity against Na_{V}1.5 and Na_{V}1.6.

### BRIEF DESCRIPTION OF THE DRAWINGS

FIG. 1 shows the effect of Peptide 1 (SEQ. ID. NO: 1) on HEK-Na_{V}1.7 stable cells. Voltage-dependent inward currents for HEK-Nav1.6 cells were evoked by depolarizations to test potentials between −100 mV and +60 mV from a holding potential of −70 mV followed by a voltage pre-step pulse of −120 mV (Na_{V}1.7). Steady-state (fast) inactivation of Na_{V} channels was measured with a paired-pulse protocol. From the holding potential, cells were stepped to varying test potentials between −120 mv (Na_{V}1.7) and +20 mV (pre-pulse) prior to a test pulse to −20 mV.

FIG. 2 shows the effect of Positive Control Peptide 2 (GpTx-1: DCLGFMRKCIPDNDKCCRPNLVCSRTHKWCKYVF) on HEK-Na_{V}1.7 stable cells. Voltage-dependent inward currents for HEK-Nav1.6 cells were evoked by depolarizations to test potentials between −100 mV and +60 mV from a holding potential of −70 mV followed by a voltage pre-step pulse of −120 mV (Na_{V}1.7). Steady-state (fast) inactivation of Na_{V} channels was measured with a paired-pulse protocol. From the holding potential, cells were stepped to varying test potentials between −120 mv (Na_{V}1.7) and +20 mV (pre-pulse) prior to a test pulse to −20 mV.

FIG. 3 shows the effect of Peptide 3 (SEQ. ID. NO: 2) on HEK-Na_{V}1.7 stable cells. Voltage-dependent inward currents for HEK-Nav1.6 cells were evoked by depolarizations to test potentials between −100 mV and +60 mV from a holding potential of −70 mV followed by a voltage pre-step pulse of −120 mV (Na_{V}1.7). Steady-state (fast) inactivation of Na_{V} channels was measured with a paired-pulse protocol. From the holding potential, cells were stepped to varying test potentials between −120 mv (Na_{V}1.7) and +20 mV (pre-pulse) prior to a test pulse to −20 mV.

FIG. 4 shows the effect of Peptide 5 (SEQ. ID. NO: 4) on HEK-Na_{V}1.7 stable cells. Voltage-dependent inward currents for HEK-Nav1.6 cells were evoked by depolarizations to test potentials between −100 mV and +60 mV from a holding potential of −70 mV followed by a voltage pre-step pulse of −120 mV (Na_{V}1.7). Steady-state (fast) inactivation of Na_{V} channels was measured with a paired-pulse protocol. From the holding potential, cells were stepped to varying test potentials between −120 mv (Na_{V}1.7) and +20 mV (pre-pulse) prior to a test pulse to −20 mV.

FIG. 5 shows the effect of Peptide 6 (SED ID. NO: 5) on HEK-Na_{V}1.7 stable cells. Voltage-dependent inward currents for HEK-Nav1.6 cells were evoked by depolarizations to test potentials between −100 mV and +60 mV from a holding potential of −70 mV followed by a voltage pre-step pulse of −120 mV (Na_{V}1.7). Steady-state (fast) inactivation of Na_{V} channels was measured with a paired-pulse protocol. From the holding potential, cells were stepped to varying test potentials between −120 mv (Na_{V}1.7) and +20 mV (pre-pulse) prior to a test pulse to −20 mV.

FIG. 6 shows the effect of Peptide 7 (SEQ. ID. NO: 6) on HEK-Na_{V}1.7 stable cells. Voltage-dependent inward currents for HEK-Nav1.6 cells were evoked by depolarizations to test potentials between −100 mV and +60 mV from a holding potential of −70 mV followed by a voltage pre-step pulse of −120 mV (Na_{V}1.7). Steady-state (fast) inactivation of Na_{V} channels was measured with a paired-pulse protocol. From the holding potential, cells were stepped to varying test potentials between −120 mv (Na_{V}1.7) and +20 mV (pre-pulse) prior to a test pulse to −20 mV.

FIG. 7 shows the effect of Peptide 8 (SEQ. ID. NO: 7) on HEK-Na_{V}1.7 stable cells. Voltage-dependent inward currents for HEK-Nav1.6 cells were evoked by depolarizations to test potentials between −100 mV and +60 mV from a holding potential of −70 mV followed by a voltage pre-step pulse of −120 mV (Na_{V}1.7). Steady-state (fast) inactivation of Na_{V} channels was measured with a paired-pulse protocol. From the holding potential, cells were stepped to varying test potentials between −120 mv (Na_{V}1.7) and +20 mV (pre-pulse) prior to a test pulse to −20 mV.

FIG. 8 shows the effect of Positive Control Peptide 9 (JzTx-V: YCQKWMWTCDSKRACCEGLRCKLWCRKII) on HEK-Na_{V}1.7 stable cells. Voltage-dependent inward currents for HEK-Nav1.6 cells were evoked by depolarizations to test potentials between −100 mV and +60 mV from a holding potential of −70 mV followed by a voltage pre-step pulse of −120 mV (Na_{V}1.7). Steady-state (fast) inactivation of Nar channels was measured with a paired-pulse protocol. From the holding potential, cells were stepped to varying test potentials between −120 mv (Na_{V}1.7) and +20 mV (pre-pulse) prior to a test pulse to −20 mV.

FIG. 9 shows the effect of Peptide 10 (SEQ. ID. NO: 8) on HEK-Na_{V}1.7 stable cells. Voltage-dependent inward currents for HEK-Nav1.6 cells were evoked by depolarizations to test potentials between −100 mV and +60 mV from a holding potential of −70 mV followed by a voltage pre-step pulse of −120 mV (Na_{V}1.7). Steady-state (fast) inactivation of Na_{V} channels was measured with a paired-pulse protocol. From the holding potential, cells were stepped to varying test potentials between −120 mv (Na_{V}1.7) and +20 mV (pre-pulse) prior to a test pulse to −20 mV.

FIG. 10 shows the effect of Peptide 11 (SEQ. ID. NO: 9) on HEK-Na_{V}1.7 stable cells. Voltage-dependent inward currents for HEK-Na_{V}1.6 cells were evoked by depolarizations to test potentials between −100 mV and +60 mV from a holding potential of −70 mV followed by a voltage pre-step pulse of −120 mV (Na_{V}1.7). Steady-state (fast) inactivation of Na_{V} channels was measured with a paired-pulse protocol. From the holding potential, cells were stepped to varying test potentials between −120 mv (Na_{V}1.7) and +20 mV (pre-pulse) prior to a test pulse to −20 mV.

FIG. 11 shows that Peptide 1 (SEQ. ID. NO. 1) has no effects on modulation of Na_{V}1.5 currents. A, representative traces of voltage-gated Na+ currents (I_{Na+}) recorded from HEK-Na_{V}1.5 cells in response to voltage steps from −120 mV to +60 mV from a holding potential of −70 mV (inset). HEK-Na_{V}1.5 cells were treated with 500 nM peptide 1 (orange) and control (black). B, current-voltage relationships of I_{Na+} from the experimental groups described in A. C, bar graph representing peak current densities measured in individual cells HEK-Na_{V}1.5 cells (control; black and 500 nM peptide 1; orange). Data are mean±SEM. SEMs are shown as error bars in the figures. NS=non-significant. Student's t test.

FIG. 12 shows that Peptide 3 (SEQ. ID. NO. 2) suppressed Na_{V}1.5 currents. A, representative traces of voltage-gated Na currents (I_{Na+}) recorded from HEK-Na_{V}1.5 cells in response to voltage steps from −120 mV to +60 mV from a holding potential of −70 mV (inset). HEK-Na_{V}1.5 cells were treated with 500 nM peptide 3 (magenta) and control (black). B, current-voltage relationships of I_{Na+} from the experimental groups described in A. C, bar graph representing peak current densities measured in individual cells HEK-Na_{V}1.5 cells (control; black and 500 nM peptide 3; magenta). Data are mean±SEM. SEMs are shown as error bars in the figures. **P<0.005; NS=non-significant. Student's t test.

FIG. 13 shows that Peptide 7 (SEQ. ID. NO. 6) has no effects on modulation of Na_{V}1.5 currents. A, representative traces of voltage-gated Na⁺ currents (I_{Na+}) recorded from HEK-Na_{V}1.5 cells in response to voltage steps from −120 mV to +60 mV from a holding potential of −70 mV (inset). HEK-Na_{V}1.5 cells were treated with 500 nM peptide 7 (green) and control (black). B, current-voltage relationships of INa+ from the experimental groups described in A. C, bar graph representing peak current densities measured in individual cells HEK-Na_{V}1.5 cells (control; black and 500 nM peptide 7; green). Data are mean±SEM. SEMs are shown as error bars in the figures. NS=non-significant. Student's t test.

FIG. 14 shows that Peptide 8 (SEQ. ID. NO. 7) has no effects on modulation of Na_{V}1.5 currents. A, representative traces of voltage-gated Na+ currents (INa+) recorded from HEK-Na_{V}.5 cells in response to voltage steps from −120 mV to +60 mV from a holding potential of −70 mV (inset). HEK-Na_{V}1.5 cells were treated with DMSO (black) or with 500 nM peptide 8 (blue). B, current-voltage relationships of I_{Na+} from the experimental groups described in A. C, bar graph representing peak current densities measured in individual cells HEK-Na_{V}1.5 cells (treated with DMSO; black and 500 nM peptide 8; blue). Data are mean±SEM. SEMs are shown as error bars in the figures. NS=non-significant. Student's t test.

FIG. 15 shows that Peptide 1 (SEQ. ID. NO. 1) suppressed Na_{V}1.6 currents. A, representative traces of voltage-gated Na+ currents (I_{Na+}) recorded from HEK-Na_{V}1.6 cells in response to voltage steps from −120 mV to +60 mV from a holding potential of −70 mV (inset). HEK-Na_{V}1.6 cells were treated with 500 nM peptide 1 (orange) and control (black). B, current-voltage relationships of I_{Na+} from the experimental groups described in A. C, bar graph representing peak current densities measured in individual cells HEK-Na_{V}1.6 cells (control; black and 500 nM peptide 1; orange). Data are mean SEM. SEMs are shown as error bars in the figures. **P<0.005 (p=0.0012). Student's t test.

FIG. 16 shows that Peptide 3 (SEQ. ID. NO. 2) suppressed Na_{V}1.6 currents. A, representative traces of voltage-gated Na+ currents (INa+) recorded from HEK-Na_{V}1.6 cells in response to voltage steps from −120 mV to +60 mV from a holding potential of −70 mV (inset). HEK-Na_{V}1.6 cells were treated with 500 nM peptide 1 (orange) and control (black). B, current-voltage relationships of I_{Na+} from the experimental groups described in A. C, bar graph representing peak current densities measured in individual cells HEK-Na_{V}1.6 cells (control; black and 500 nM peptide 1; orange). Data are mean±SEM. SEMs are shown as error bars in the figures. **P<0.005 (p=0.0012). Student's t test.

FIG. 17 shows that Peptide 7 (SEQ. ID. NO. 6) potentiates the Na_{V}1.6 currents. A, representative traces of voltage-gated Na+ currents (INai) recorded from HEK-Na_{V}1.6 cells in response to voltage steps from −120 mV to +60 mV from a holding potential of −70 mV (inset). HEK-Na_{V}1.6 cells were treated with 500 nM peptide 7 (green) and control (black). B, current-voltage relationships of I_{Na+} from the experimental groups described in A. C, bar graph representing peak current densities measured in individual cells HEK-Na_{V}1.6 cells (control; black and 500 nM peptide 7; green). Data are mean±SEM. SEMs are shown as error bars in the figures. **P<0.005 (p=0.0048). Student's t test.

FIG. 18 shows that Peptide 8 (SEQ. ID. NO. 7) has no effects on modulation of Na_{V}1.6 currents. A, representative traces of voltage-gated Na+ currents (INa+) recorded from HEK-Na_{V}1.6 cells in response to voltage steps from −120 mV to +60 mV from a holding potential of −70 mV (inset). HEK-Na_{V}1.6 cells were treated with DMSO (black) or with 500 nM peptide 8 (blue). B, current-voltage relationships of I_{Na+} from the experimental groups described in A. C, bar graph representing peak current densities measured in individual cells HEK-Nav1.6 cells (treated with DMSO; black and 500 nM peptide 8; blue). Data are mean±SEM. SEMs are shown as error bars in the figures. NS=non-significant. Student's t test.

### DETAILED DESCRIPTION

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. Thus, as used in this specification and the appended claims, the singular forms “a”, “an” and “the” include plural referents unless the context clearly indicates otherwise. For example, reference to “a peptide” includes a plurality of peptides.

As used herein, the term “peptide” refers to a compound consisting of two or more amino acid residues linked in a chain via peptide bonds such that the carboxyl group of the amino acid residue is joined to the amino group of the adjacent amino acid residue to form a —CO—NH— bond.

As used herein, the term “amino acid residue” refers to a specific amino acid, usually dehydrated as a result of its involvement in two peptide bonds or in a polypeptide backbone, but also when the amino acid is involved in one peptide bond, as occurs at each end of a linear polypeptide chain. The amino acid residues may be referred to by the commonly accepted three-letter codes or single-letter codes frequently applied to designate the identifies of the twenty “canonical” amino acid residues generally incorporated into naturally occurring peptides and proteins (Table 2). Such one-letter abbreviations are entirely interchangeable in meaning with three-letter abbreviations, or non-abbreviated amino acid names. Within the one-letter abbreviation system used herein, an uppercase letter indicates a L-amino acid, and a lower case letter indicates a D-amino acid. For examples. the abbreviation “R” designates L-arginine and the abbreviation “r” designates D-arginine.

TABLE 2

| Amino Acid | Three Letter Code | One Letter Code |
|---|---|---|
| Alanine | Ala | A |
| Glutamine | Gln | Q |
| Leucine | Leu | L |
| Serine | Ser | S |
| Arginine | Arg | R |
| Glutamic Acid/Glutamate | Glu | E |
| Lysine | Lys | K |
| Threonine | Thr | T |
| Asparagine | Asn | N |
| Glycine | Gly | G |
| Methionine | Met | M |
| Tryptophan | Trp | W |
| Aspartic Acid/Aspartate | Asp | D |
| Histidine | His | H |
| Phenylalanine | Phe | F |
| Tyrosine | Tyr | Y |
| Cysteine | Cys | C |
| Isoleucine | Ile | I |
| Proline | Pro | P |
| Valine | Val | V |

Non-canonical amino acid residues can be incorporated into a peptide within the scope of the invention by employing known synthetic techniques or known techniques of protein engineering. The term “non-canonical amino acid residue” refers to amino acid residues in D- or L-form that are not among the 20 canonical amino acids generally incorporated into naturally occurring proteins, for example, β-amino acids, homoamino acids, cyclic amino acids and amino acids with derivatized side chains. Non-limiting examples of non-canonical amino acid residues include (in the L-form or D-form) β-alanine, β-aminopropionic acid, piperidinic acid, aminocaprioic acid, aminoheptanoic acid, aminopimelic acid, desmosine, diaminopimelic acid, N^{α}-ethylglycine, N^{α}-ethylaspargine, hydroxylysine, allo-hydroxylysine. isodesmosine, allo-isoleucine, ω-methylarginine, N^{α}-methylglycine, N^{α}-methylisoleucine, N^{α}-methylvaline, γ-carboxyglutamate, ε-N,N,N-trimethyllysine, ε-N-acetyl-lysine, O-phosphoserine, N^{α}-acetylserine, N^{α}-formylmethionine, 3-methylhistidine, 5-hydroxylysine, and other similar amino acids. Nomenclature and symbolism for amino acids and peptides by the UPAC-IUB Joint Commission on Biochemical Nomenclature (JCBN) have been published in the following documents: Biochem. J. 219:345-373 (1984); Eur: J. Biochem. 138:9-37 (1984); Internat. J. Pept. Prot. Res. 24, 84 (1984); J. Biol. Chem. 260: 14-42 (1985); and Amino Acids and Peptides 16:387-410 (1985).

As used herein, a “prodrug” of a peptide of the present disclosure is converted to the peptide of the present disclosure via an enzymatic reaction, typically under physiological conditions in the living body, that is, conversion from the prodrug to the peptide occurs by enzymatically catalyzed oxidation, reduction, or hydrolysis, etc. Methods for making peptide prodrugs are known in the art. For example, see Oliyai, R., Adv. Drug Delivery Rev. 1996, 19, 275-286; Oliyai et al., Ann. Rev. Pharmcol. Toxicol. 1993 32, 521-44; Paulette et al. Adv. Drug Delivery Rev. 1997, 27, 235-256.

The peptides disclosed herein are peptides having a sequence that differ from a peptide sequence existing in nature by at least one amino acid residue substitution, internal addition, or internal deletion of at least one amino acid, and/or amino- or carboxy-terminal end truncations or additions, and/or carboxy-terminal amidation. An “internal deletion” refers to absence of an amino acid from a sequence existing in nature at a position other than the N- or C-terminus. Likewise, an “internal addition” refers to presence of an amino acid in a sequence existing in nature at a position other than the N- or C-terminus. The peptides disclosed herein, and the pharmaceutically acceptable salts, prodrugs and solvates thereof, are useful as blockers of sodium (Na⁺) channels, and particularly Na_{V}1.7 channels. These peptides of the present disclosure show selectivity as Na_{V}1.7 channel blockers in the absence of disulfide structures.

In certain embodiments of the inventive composition of matter are derived from a toxin peptide and contain modifications of a native toxin peptide sequence of interest (e g, amino acid residue substitutions, internal additions or insertions, internal deletions, and/or amino- or carboxy-terminal end truncations, or additions as previously described above) relative to a native toxin peptide sequence of interest, such as JzTx-V (YCQKWMWTCDSKRACCEGLRCKLWCRKII (Peptide 9)) or GpTx-1 (DCLGFMRKCIPDNDKCCRPNLVCSRTHKWCKYVF (Peptide 2)). In certain embodiments said modifications are identified in silico using a bioinformatics platform similar to the methods discussed in Velijkovic, et al. PLOS ONE, Nov. 9, 2016. In contrast, substantial modifications in the functional and/or chemical characteristics of peptides may be accomplished by selecting substitutions in the amino acid sequence that differ significantly in their effect on maintaining (a) the structure of the molecular backbone in the region of the substitution, for example, as an α-helical conformation, (b) the charge or hydrophobicity of the molecule at the target site, or (c) the size of the molecule. For example, a “conservative amino acid substitution” may involve a substitution of a native amino acid residue with a nonnative residue such that there is little or no effect on the polarity or charge of the amino acid residue at that position. Furthermore, any native residue in the polypeptide may also be substituted with alanine, as has been previously described for “alanine scanning mutagenesis” (see, for example, MacLennan et al., Acta Physiol. Scand Suppl., 643:55-67 (1998), which discusses alanine scanning mutagenesis).

In one embodiment, the present invention is directed to a composition of matter comprising of a peptide comprising the amino acid sequence SEQ ID NO 1: SDEIPATFGGGTDAGL (Peptide 1).

In one embodiment, the present invention is directed to a composition of matter comprising of a peptide comprising the amino acid sequence SEQ ID NO 2: DCLGLFRKCIPDMLKCCRFNLVCSRLHKWCKYVF (Peptide 3).

In one embodiment, the present invention is directed to a composition of matter comprising of a peptide comprising the amino acid sequence SEQ ID NO. 3: DCLGLFRKCIPDNDKCCRPNFVCSRTHKVCFYVL (Peptide 4).

In one embodiment, the present invention is directed to a composition of matter comprising of a peptide comprising the amino acid sequence SEQ ID NO. 4: DCLGLFRKCIPDNDKCCRPNLVCSRTHKVCFYVL (Peptide 5).

In one embodiment, the present invention is directed to a composition of matter comprising of a peptide comprising the amino acid sequence SEQ ID NO 5: DCLGMFRKCLPDDDKCCRPNLVCSRTHKWCRLVL (Peptide 6).

In one embodiment, the present invention is directed to a composition of matter comprising of a peptide comprising the amino acid sequence SEQ ID NO 6: IGEKVTIRLITSTNINDDFNIYQQKPGEPPKLL (Peptide 7).

In one embodiment, the present invention is directed to a composition of matter comprising of a peptide comprising the amino acid sequence SEQ ID NO 7: DCLGFMRKCLSTTDLDDDWNCCRPNLVCSRTHKWCKYVF (Peptide 8).

In one embodiment, the present invention is directed to a composition of matter comprising of a peptide comprising the amino acid sequence SEQ ID NO 8: YCQRFMLTCDSKKACCEGLRCKLLCRKII (Peptide 10).

In one embodiment, the present invention is directed to a composition of matter comprising of a peptide comprising the amino acid sequence SEQ ID NO 9:YCQRWLWTCDSKKACCEGLRCKLWCRKII (Peptide 11).

In one embodiment, the present invention is directed to a composition of matter comprising of a peptide comprising the amino acid sequence SEQ ID NO 10: YCLGFMRKCDSERKCCEGMVCRLWCKRRLW (Peptide 12).

In one embodiment, the present invention is directed to a composition of matter comprising of a peptide comprising the amino acid sequence SEQ ID NO 11: SCRRAWMACDTAKVCCNPIKCRVACKRVL (Peptide 13).

In particular embodiments. the composition of matter comprises an amino acid sequence selected from SEQ ID NOs: 1-11; or comprises an amino acid sequence selected from SEQ ID NOs: 1-11, that does not include anon-canonical amino acid.

The present invention also encompasses a nucleic acid (e.g., DNA or RNA) encoding any of SEQ ID NOS: 1-11, that does not include a non-canonical amino acid; an expression vector comprising the nucleic acid: and a recombinant host cell comprising the expression vector.

Peptide compositions of the present disclosure can be made by synthetic methods. Solid phase synthesis is the preferred technique of making individual peptides since it is the most cost-effective method of making small peptides. For example, well known solid phase synthesis techniques include the use of protecting groups, linkers, and solid phase supports, as well as specific protection and deprotection reaction conditions, linker cleavage conditions, use of scavengers, and other aspects of solid phase peptide synthesis. Suitable techniques are well known in the art. (E.g., Merrifield (1973), Chem. Polypeptides, pp. 335-61 (Katsoyannis and Panayotis eds.); Merrifield (1963), J. Am. Chem. Soc. 85: 2149; Davis et al. (1985), Biochem. Intl. 10: 394-414. Further examples of synthetic and purification methods known in the art, which may be applicable to making the inventive compositions of matter include, Sullivan et al., Toxin Peptide Therapeutic Agents, US2007/0071764 and Sullivan et al., Toxin Peptide Therapeutic Agents, PCT/US2007/022831, published as WO 2008/088422 A2.

Recombinant DNA- and/or RNA-mediated protein expression and protein engineering techniques, or any other methods of preparing peptides, are applicable to the making of the inventive peptides. For example, the peptides can be made in transformed host cells. Briefly, a recombinant DNA molecule, or construct, coding for the peptide is prepared. Methods of preparing such DNA molecules are well known in the art. For instance, sequences encoding the peptides can be excised from DNA using suitable restriction enzymes. Any of a large number of available and well-known host cells may be used in the practice of this invention. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. Useful microbial host cells in culture include bacteria (such as Escherichia coli sp.), yeast (such as Saccharomyces sp.) and other fungal cells, insect cells, plant cells, mammalian (including human) cells, e.g., CHO cells and HEK293 cells. Modifications can be made at the DNA level, as well. The peptide-encoding DNA sequence may be changed to codons more compatible with the chosen host cell. For E. coli, optimized codons are known in the art. Codons can be substituted to eliminate restriction sites or to include silent restriction sites, which may aid in processing of the DNA in the selected host cell. Next, the transformed host is cultured and purified. Host cells may be cultured under conventional fermentation conditions so that the desired compounds are expressed. Such fermentation conditions are well known in the art.

The present disclosure further provides pharmaceutical compositions comprising an effective amount of a peptide comprising of an amino acid sequence selected from the group consisting of SEQ ID NOs 1-11, or a pharmaceutically acceptable salt, prodrug or solvate thereof, in a mixture with one or more pharmaceutically acceptable carriers. Pharmaceutical compositions of the present disclosure are useful for treating or preventing a disorder responsive to the blockade of sodium ion channels, especially Na_{V}1.7 sodium ion channels.

Examples of pharmaceutically acceptable addition salts include inorganic and organic acid addition salts and basic salts. The pharmaceutically acceptable salts include, but are not limited to, metal salts such as sodium salt, potassium salt, cesium salt and the like: alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like: inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate or the like. Acid addition salts can be formed by mixing a solution of the particular peptide of the invention with a solution of a pharmaceutically acceptable non-toxic acid such as hydrochloric acid, fumaric acid, maleic acid, succinic acid, acetic acid, citric acid, tartaric acid, carbonic acid, phosphoric acid, oxalic acid, dichloroacetic acid, or the like. Basic salts can be formed by mixing a solution of the peptide selected from the group comprising of SEQ. tD NOs: 1-11 with a solution of a pharmaceutically acceptable non-toxic base such as sodium hydroxide, potassium hydroxide, choline hydroxide, sodium carbonate or the like.

The embodiments disclosed herein are also meant to encompass solvates of any of the disclosed peptides. Solvates typically do not significantly alter the physiological activity or toxicity of the peptides, and as Such may function as pharmacological equivalents. The term “solvate” as used herein is a combination, physical association and/or solvation of a peptide of the invention with a solvent molecule Such as, e.g. a disolvate, monosolvate or hemisolvate, where the ratio of solvent molecule to peptide is typically 2:1, 1:1 or 1:2, respectively. This physical association involves varying degrees of ionic and covalent bonding, including hydrogen bonding. In certain instances, the solvate can be isolated. Such as when one or more solvent molecules are incorporated into the crystal lattice of a crystalline solid. Thus, “solvate” encompasses both solution-phase and isolatable solvates. Peptides of the present disclosure may be unsolvated, or may be solvated with a pharmaceutically acceptable solvent such as water, methanol, ethanol, and the like. One type of solvate is a hydrate. A “hydrate” relates to a particular subgroup of solvates where the solvent molecule is water. Methods for preparing solvates are generally known in the art. See, for example, M. Caira et al., J. Pharmaceut. Sci., 93(3):601-611 (2004), which describes the preparation of Solvates of fluconazole with ethyl acetate, and with water. Similar preparation of solvates, hemisolvates, hydrates, and the like are described by E. C. van Tonder et al., AAPS Pharm. Sci. Tech., 5(1): Article 12 (2004), and A. L. Bingham et al., Chem. Commun. 603-604 (2001). A typical, non-limiting, process of preparing a solvate would involve dissolving a peptide of the invention in a desired solvent (organic, water, or a mixture thereof) at temperatures above about 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

The present disclosure further provides methods of treating a disorder responsive to the blockade of sodium channels, and particularly Na_{V}1.7 sodium channels, in a mammal suffering from excess activity of said channels, said methods comprising administering to said mammal an effective amount of a peptide comprising the amino acid sequence selected from the group comprising of SEQ ID NOs:1-11, or a pharmaceutically acceptable salt, prodrug or solvate thereof, as described herein. In a preferred embodiment, the disorder being treated is pain (e.g., acute pain, chronic pain, or inflammatory pain, which includes but is not limited to, neuropathic pain and surgical pain).

The present disclosure further provides the use of a peptide comprising the amino acid sequence selected from the group comprising of SEQ ID NOs: 1-11, or a pharmaceutically acceptable salt, prodrug or solvate thereof, in the manufacture of a medicament useful to treat or prevent a disorder responsive to the blockade of sodium channels, and particularly Na_{V}1.7 sodium channels. In a preferred embodiment, the disorder being treated or prevented is pain (e.g., acute pain, chronic pain, or inflammatory pain, which includes but is not limited to, neuropathic pain and surgical pain).

In one embodiment, the present invention provides a method of treating pain (palliative treatment). In another embodiment, the present invention provides a method of preventing pain (pre-emptive treatment). In one embodiment, the type of pain treated is chronic pain. In another embodiment, the type of pain treated is acute pain. In another embodiment, the type of pain treated is neuropathic pain. In another embodiment, the type of pain treated is inflammatory pain. In another embodiment, the type of pain treated is surgical pain. In each instance, Such method of treatment or prevention requires administering to a subject in need of such treatment or prevention an amount of a peptide of the invention that is therapeutically effective in achieving said result. In one embodiment, the amount of such peptide is the amount that is effective to substantially block sodium channels in vivo.

Chronic pain includes, but is not limited to, inflammatory pain, neuropathic pain, postoperative pain, cancer pain, osteoarthritis pain associated with metastatic cancer, trigeminal neuralgia, acute herpetic and postherpetic neuralgia, diabetic neuropathy, causalgia, brachial plexus avulsion, occipital neuralgia, reflex sympathetic dystrophy, fibromyalgia, gout, phantom limb pain, burn pain, and other forms of neuralgia, neuropathic, and idiopathic pain syndromes.

The methods of the present invention may be used to treat or prevent chronic Somatic pain, which generally results from inflammatory responses to tissue injury Such as nerve entrapment. Surgical procedures, cancer or arthritis (Brower, Nature Biotechnology 2000; 18:387-391). Inflammatory pain includes, but is not limited to, pain associated with osteoarthritis and rheumatoid arthritis.

The present invention further provides a method of modulating the activity of sodium ion channels, especially Na_{V}1.7 sodium ion channels, in a cell, or in a membrane preparation, which method comprises administering to the cell or membrane preparation an effective amount of a peptide comprising the amino acid sequence selected from the group comprising of SEQ ID NOs: 1-11, or a pharmaceutically acceptable salt, prodrug or solvate thereof. In certain embodiments, the method is carried out in an in vitro cellular or membrane assay system. In other embodiments, the method is carried out in an in vivo system, e.g., in a mammal such as a human.

The methods of the present invention may also be used to treat or prevent epilepsy, seizures, epilepsy with febrile seizures, epilepsy with benign familial neonatal infantile seizures, inherited pain disorders, e.g., primary erythermalgia and paroxysmal extreme pain disorder, familial hemiplegic migraine, movement disorder, psychiatric disorders (such as autism, cerebeller atrophy, ataxia, and mental retardation/neurodegeneration), global or focal ischemia, myotonia, a movement disorder, erythermalgia, cardiac arrhythmias or other conduction disorders, including Supraventricular tachy cardia, Ventricular tachycardia, symptomatic ventricular premature beats, and prevention of ventricular fibrillationventricular fibrillation, and to provide local anesthesia.

The compositions of the invention provide an effective method of treating, or preventing, pain, for example acute, persistent, or chronic pain. Selectivity against off-target sodium channels. In some embodiment the compositions of the invention provide selectivity against those Na_{V} channels governing cardiac excitability (Na_{V}1.5) and skeletal muscle excitability (Na_{V}1.4), is cardinal for any systemically delivered therapeutic. In other embodiments compositions of the present invention provide such selectivity against Na_{V} 1.5 and Na_{V}1.6.

In one embodiment, the subject being treated by a method of the present invention is a mammal. In another embodiment, the mammal is a human, or other primate (e.g., a chimpanzee, orangutan, gorilla, or lemur), or a canine (e.g., a dog, fox, wolf, or coyote), feline (e.g., a cat, lion, tiger, bobcat, leopard, cheetah, panther), equine (e.g., a horse, llama, alpaca, zebra, deer, moose, elk, mule or donkey), bovine (e.g., a cow, a bull, a buffalo or a bison), or a pig, marine mammal (e.g., a seal, walrus, otter, sea lion, manatee, dolphin, porpoise or whale), rodent (e.g., a rat, a mouse, ferret or guinea pig), or any other mammal.

In one embodiment, a peptide of the invention is administered to the subject by any suitable route of administration, including by one or more of the oral, buccal, mucosal, sublingual, parenteral, subcutaneous, intramuscular, intraperitoneal, intrathecal, intranasal, inhalation, transdermal, rectal or vaginal routes of administration.

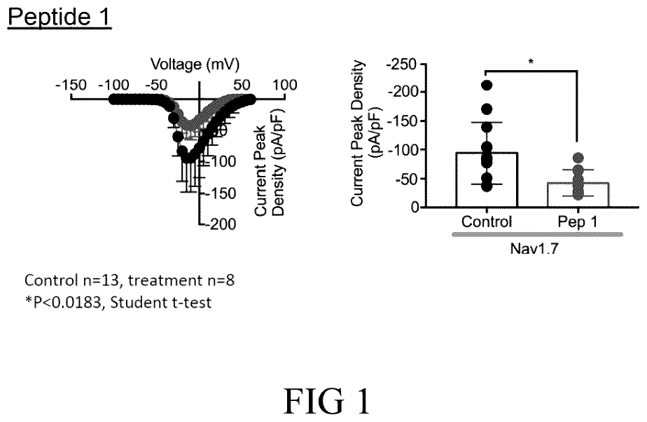

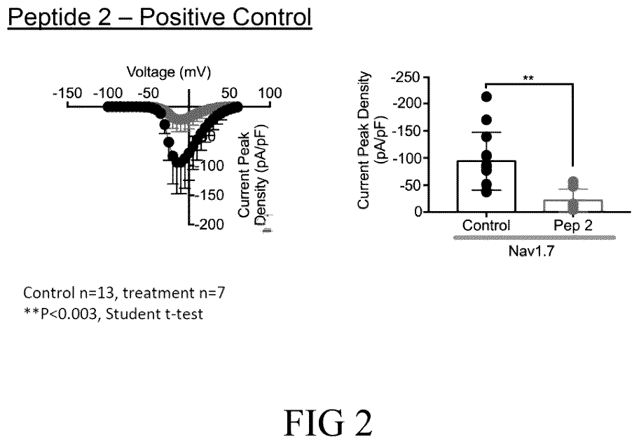

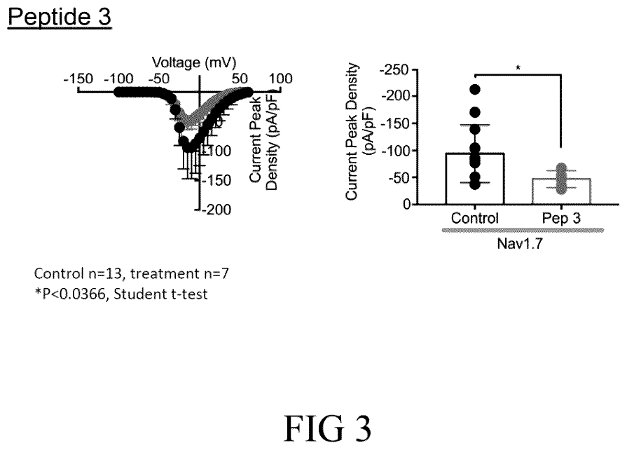

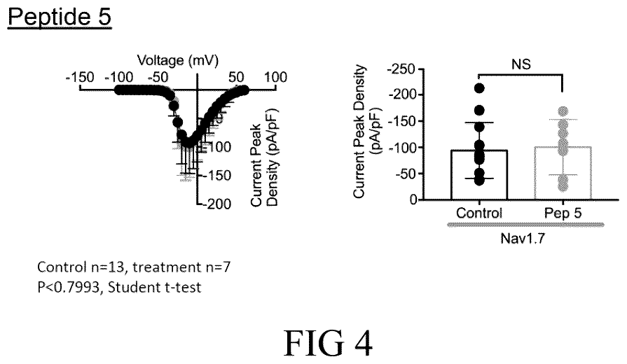

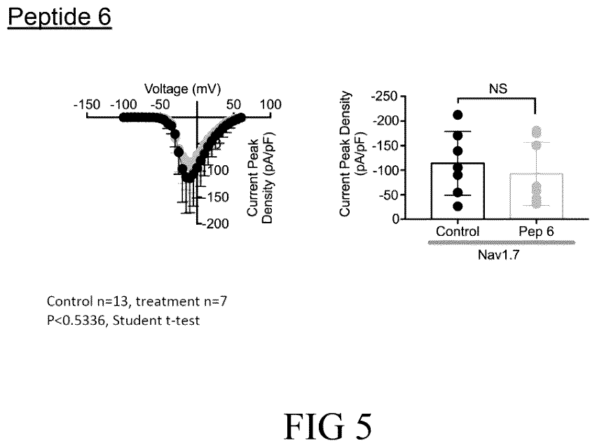

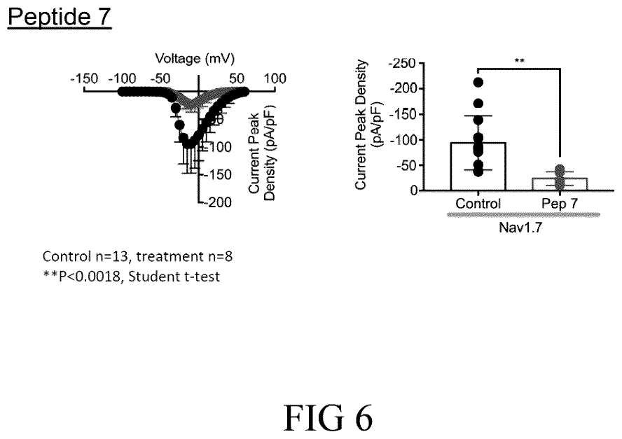

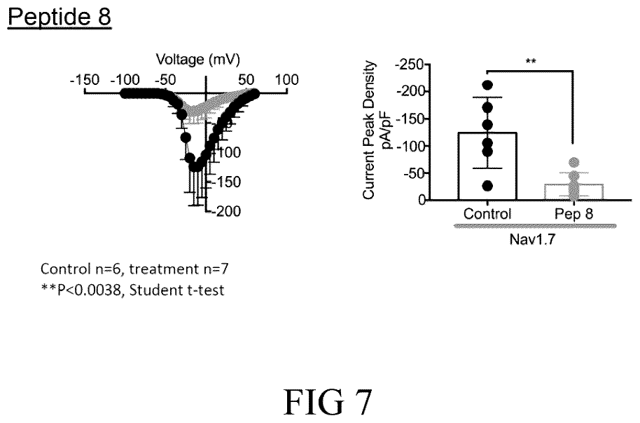

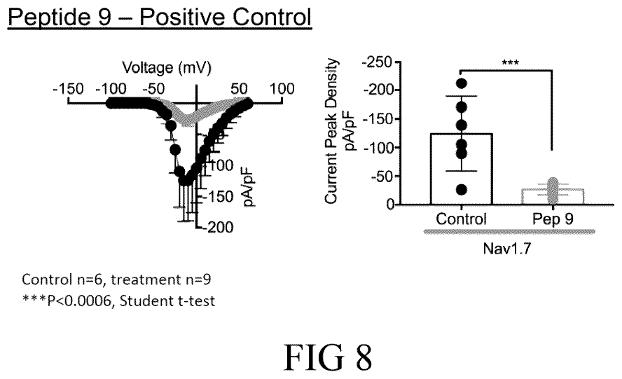

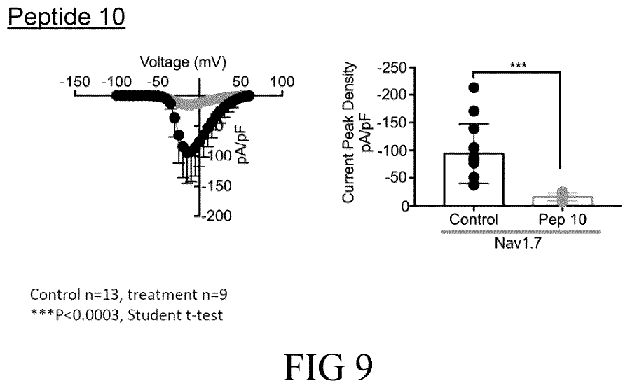

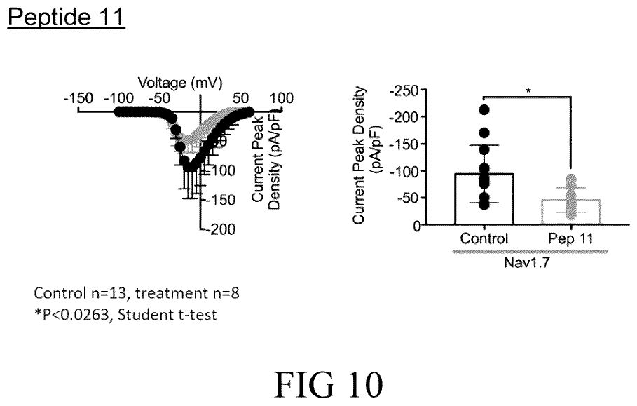

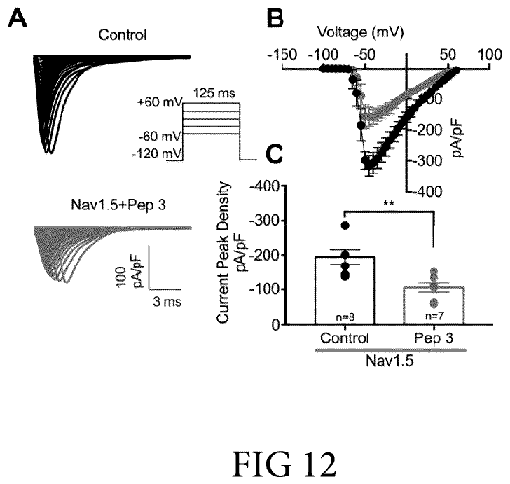

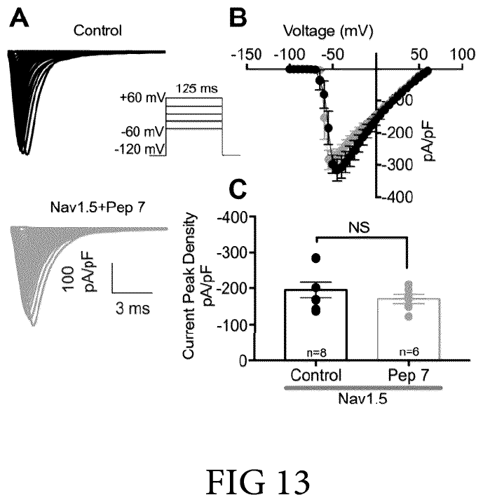

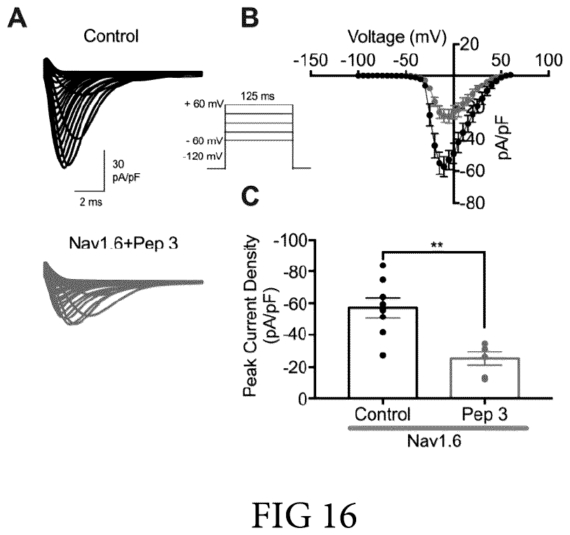

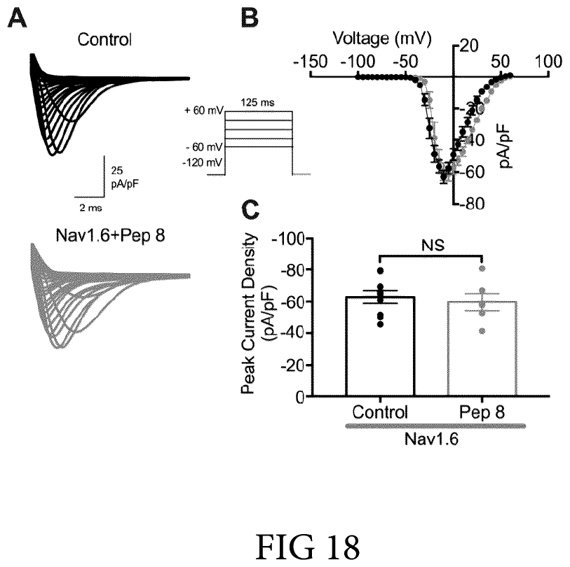

As shown in FIGS. 1-18, the representative peptides of the present disclosure can be assessed by electrophysiological assays testing for sodium channel activity. One aspect of the present disclosure is based on the use of the peptides herein described as sodium channel blockers. In certain embodiments of the present disclosure, it has been found that certain peptides show selectivity as Na_{V}1.7 sodium channel blockers. Based upon this property, these peptides are considered useful in treating pain.

More specifically, the present disclosure is directed to peptides that are blockers of sodium channels. In one embodiment, peptides having preferred sodium channel blocking properties exhibit an IC₅₀ of about 100 μM or less in one or more of the sodium electrophysiological assays described herein, or an IC₅₀ of 10 μM or less, or an IC₅₀ of about 6 μM or less, or an IC₅₀ of about 1.0 μM or less, or an IC₅₀ of about 500 nM or less, or an IC₅₀ if about 100 nM or less.

In certain embodiments, peptides useful in the present invention are those represented by SEQ ID NOs: 1-11 that exhibit selectivity for Na_{V}1.7 sodium channels over Na_{V}1.5 sodium channels in electrophysiological assays described herein. The phrase “selectivity for Na_{V}1.7 sodium channels over Na_{V}1.5 sodium channels is used herein to mean that the ratio of an IC₅₀ for Na_{V} 1.7 sodium channel blocking activity for a peptide of the invention over an IC₅₀ for Na_{V}1.5 sodium channel blocking activity for the same peptide is less than 1, i.e., Na_{V}1.7 IC₅₀/Na_{V}1.5 IC₅₀<1. Preferably, a peptides of SEQ. ID. NOs.: 1-11 exhibits an Na_{V} 1.7 IC₅₀/Na_{V}1.5 IC₅₀ ratio of about 1/2, 1/3, 1/4, 1/5, 1/6, 1/7, 1/8, 1/9, 1/10, 1/15, 1/20, 1/25, 1/30, 1/35, 1/40, 1/45, 1/50, 1/55, 1/60, 1/65, 1/70, 1/75, 1/80, 1/85, 1/90, 1/95, 1/100, 1/125, 1/150, 1/175, 1/200, 1/225, 1/250, 1/275, 1/300, 1/325, 1/350, 1/375, 1/400, 1/425, 1/450, 1/475 or 1/500 or less.

In certain embodiments, peptides useful in the present invention are those represented by SEQ ID NOs: 1-11 that exhibit selectivity for Na_{V}1.7 sodium channels over Na_{V}1.4 sodium channels in electrophysiological assays described herein. Preferably, a peptides of SEQ. ID. NOs.: 1-11 exhibits an Na_{V} 1.7 IC₅₀/Na_{V}1.4 IC₅₀ ratio of about 1/2, 1/3, 1/4, 1/5, 1/6, 1/7, 1/8, 1/9, 1/10, 1/15, 1/20, 1/25, 1/30, 1/35, 1/40, 1/45, 1/50, 1/55, 1/60, 1/65, 1/70, 1/75, 1/80, 1/85, 1/90, 1/95, 1/100, 1/125, 1/150, 1/175, 1/200, 1/225, 1/250, 1/275, 1/300, 1/325, 1/350, 1/375, 1/400, 1/425, 1/450, 1/475 or 1/500 or less.

In certain embodiments, peptides useful in the present invention are those represented by SEQ ID NOs: 1-11 that exhibit selectivity for Na_{V}1.7 sodium channels over Na_{V}1.6 sodium channels in electrophysiological assays described herein. Preferably, a peptides of SEQ. ID. NOs.: 1-11 exhibits an Na_{V} 1.7 IC₅₀/Na_{V}1.6 IC₅₀ ratio of about 1/2, 1/3, 1/4, 1/5, 1/6, 1/7, 1/8, 1/9, 1/10, 1/15, 1/20, 1/25, 1/30, 1/35, 1/40, 1/45, 1/50, 1/55, 1/60, 1/65, 1/70, 1/75, 1/80, 1/85, 1/90, 1/95, 1/100, 1/125, 1/150, 1/175, 1/200, 1/225, 1/250, 1/275, 1/300, 1/325, 1/350, 1/375, 1/400, 1/425, 1/450, 1/475 or 1/500 or less.

Peptides of the present disclosure were tested for their sodium channel blocking activity using electrophysiological assays known in the art and disclosed herein.

HEK-Na_{V}1.7 stable cell lines were plated at low density on glass cover slips for 3-4 hours and subsequently transferred to the recording chamber. Recordings were performed at room temperature (20-22° C.) 24 h post-transfection using a MultiClamp 700B amplifier (Molecular Devices, Sunnyvale, CA). The composition of recording solutions consisted of the following salts: extracellular (mM): 140 NaCl, 3 KCl, 1 MgCl₂, 1 CaCl₂), 10 HEPES, 10 glucose, pH 7.3; intracellular (mM): 130 CH₃O₃SCs, 1 EGTA, 10 NaCl, 10 HEPES, pH 7.3. Membrane capacitance and series resistance were estimated by the dial settings on the amplifier and compensated for electronically by 70-80%. Data were acquired at 20 kHz and filtered at 5 kHz prior to digitization and storage. All experimental parameters were controlled by Clampex 9.2 software (Molecular Devices) and interfaced to the electrophysiological equipment using a Digidata 1200 analog-digital interface (Molecular Devices). Voltage-dependent inward currents for HEK-Na_{V}1.7 cells were evoked by depolarizations to test potentials between −100 mV and +60 mV from a holding potential of −70 mV followed by a voltage pre-step pulse of −120 mV (Na_{V}1.7). Steady-state (fast) inactivation of Na_{V} channels was measured with a paired-pulse protocol. From the holding potential, cells were stepped to varying test potentials between −120 mv (Nar 1.7) and +20 mV (pre-pulse) prior to a test pulse to −20 mV.

Current densities were obtained by dividing Na+ current (I_{Na}) amplitude by membrane capacitance. Current-voltage relationships were generated by plotting current density as a function of the holding potential. Conductance (GNa) was calculated by the following equation:

G_{Na}=I_{Na}/(Vₘ−Eᵣₑᵥ)
where I_{Na} is the current amplitude at voltage Vₘ, and Eᵣₑᵥ is the Na⁺ reversal potential.

To test whether given peptides (10 in total, listed below) had modulatory effects on Na_{V}1.7-mediated Na⁺ currents, we used patch-clamp electrophysiology of human embryonic kidney cells stably expressing Na_{V}1.7 (HEK-Na_{V}1.7). Each group was either treated with peptide or 10 (500 nM from a stock solution dissolved in H2O or DMSO. HEK-Na_{V}1.7cells pretreated with peptide, the peak current density of Na_{V}1.7-mediated transient Na+ currents (INa+) was statistically suppressed (control H2O-64.38±16.25 and DMSO 123.99±26.62 pA/pF, n=5-6) as compared to vehicle (H2O or DMSO) treatment.

FIGS. 1-10 show the results of these studies. As can be seen the peptides of the present disclosure show blockage of the Na_{V}1.7 sodium channel. In particular peptides 1, 3, 7, and 8 exhibit substantial blockage of Na_{V}1.7 sodium channel.

Peptides of the present disclosure were tested for their selectivity in blocking Na_{V} sodium channel blocking activity using electrophysiological assays known in the art and disclosed herein.

### Cell Culture and Transient Transfections

HEK293 cells were maintained in DMEM and F-12 (Invitrogen, Carlsbad, CA), supplemented with 0.05% glucose, 0.5 mM pyruvate, 10% fetal bovine serum, 100 units/ml penicillin, and 100 μg/ml streptomycin (Invitrogen), and incubated at 37° C. with 5% CO₂. HEK293 cells stably expressing the human Na_{V}1.5 channel (hereafter referred to as HEK-Na_{V}1.5 cells) were maintained similarly except for the addition of 500 μg/ml G418 (Invitrogen) to maintain stable Na_{V} 1.5 expression. Cells were grown to 80-90% confluence, washed and re-plated at very low density prior to electrophysiological recordings.

### Electrophysiology Experiments in Heterologous Cells

HEK-Na_{V}1.6 cells were plated at low density on glass cover slips for 3-4 hours and subsequently transferred to the recording chamber. Recordings were performed at room temperature (20-22° C.) after 48 h to thaw them using a MultiClamp 200B and 700B amplifier (Molecular Devices, Sunnyvale, CA). The composition of recording solutions consisted of the following salts; extracellular (mM): 140 NaCl, 3 KCl, 1 MgCl₂, 1 CaCl₂), 10 HEPES, 10 glucose, pH 7.3; intracellular (mM): 130 CH₃O₃SCs, 1 EGTA, 10 NaCl, 10 HEPES, pH 7.3. Membrane capacitance and series resistance were estimated by the dial settings on the amplifier and compensated for electronically by 70-75%. Data were acquired at 20 kHz and filtered at 5 kHz prior to digitization and storage. All experimental parameters were controlled by Clampex 9.2 software (Molecular Devices) and interfaced to the electrophysiological equipment using a Digidata 1200 analog-digital interface (Molecular Devices). Voltage-dependent inward currents for HEK-Na_{V} 1.6 cells were evoked by depolarization to test potentials between −100 mV and +60 mV from a holding potential of −70 mV followed by a voltage pre-step pulse of −120 mV (Nar 1.6).

Current densities were obtained by dividing Na⁺ current (I_{Na}⁺) amplitude by membrane capacitance. Current-voltage relationships were generated by plotting current density as a function of the holding potential.

Statistics were calculated as mean and standard error of the mean (mean SEM) using Prism 7 (La Jolla, CA). The statistical significance of observed differences among groups was determined by Student's t-test or for two group comparisons significance was tested with unpaired. Electrophysiological data analysis was performed using Clampfit 9 software (Molecular Devices).

### Results

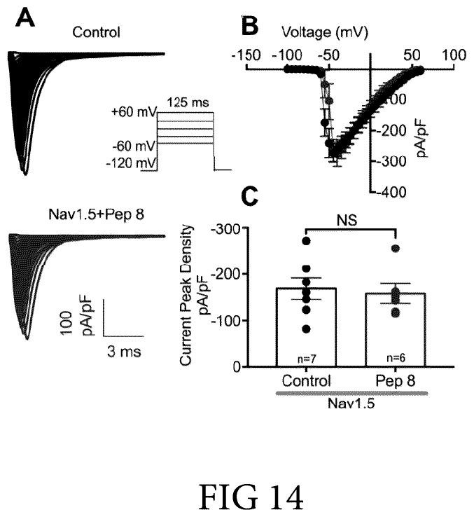

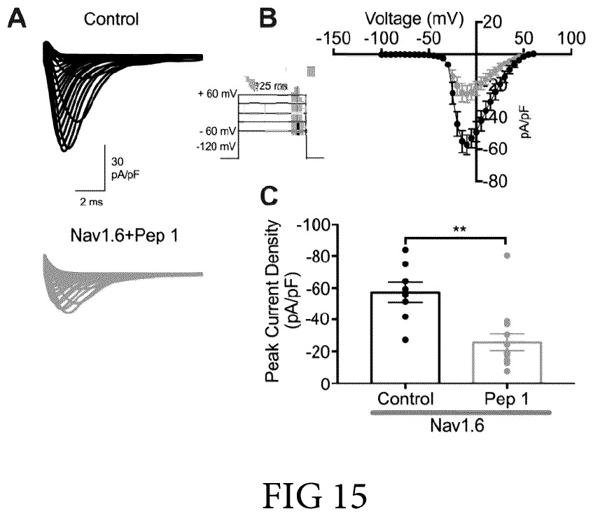

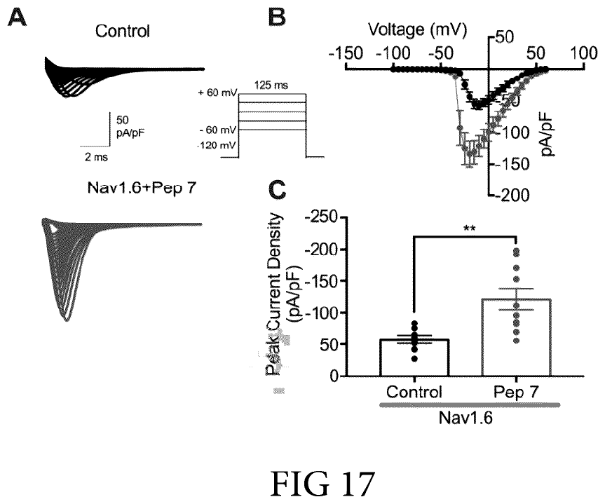

To test whether Peptide 1, 3, 7 and 8 had any modulatory effects on Na_{V} 1.6-mediated Na⁺ currents, we used whole-cell patch-clamp electrophysiology in HEK293 cells stably expressing Na_{V}1.6 (HEK-Na_{V}1.6) channels; each group was either treated with peptides 1, 3, 7 and 8 (500 nM from a stock solution dissolved in H₂O or DMSO) or H₂O and 0.1% DMSO alone (control). Except peptide 8 (control 194.86 20.9, n=8; treatment 104.78 12.9, n=7; FIG. 14A-C and Table 1) has no statistically significant effect on Na_{V}1.6 currents. Interestingly, peptide 1 & 3 showed similar effect and suppresses the Na_{V} 1.6 currents significantly compared to control (FIGS. 15 & 16, A-C and Table 1). However, peptide 7 potentiates the Na_{V}1.6-mediated transient Na⁺ currents (I_{Na+}), and were not statistically different compared to control (FIG. 17 A-C and Table 1).

TABLE 3
Effect of peptide 1, 3, 7 and 8 on Nav1.6-mediated currents

| | Peak current density | | | |
|---|---|---|---|---|
| Condition | Pep 1 | Pep 3 | Pep 7 | Pep 8 |
| Nav1.6 | 57.27 ± 6.3 (8) | 57.27 ± 6.3 (8) | 57.27 ± 6.3 (8) | 62.93 ± 4.0 (9) |
| (Control) | | | | |
| Nav1.6 | 25.65 ± 5.2 (13)^{a} | 25.32 ± 4.1 (6)^{b} | 121.14 ± 16.6 (10)^{c} | 59.75 ± 5.4 (6)^{NS} |
| (Treatment) | | | | |

^{a,b,c}p < 0.005, unpaired Student t test compared to Nav1.6 (control);
^{NS}= non-significant

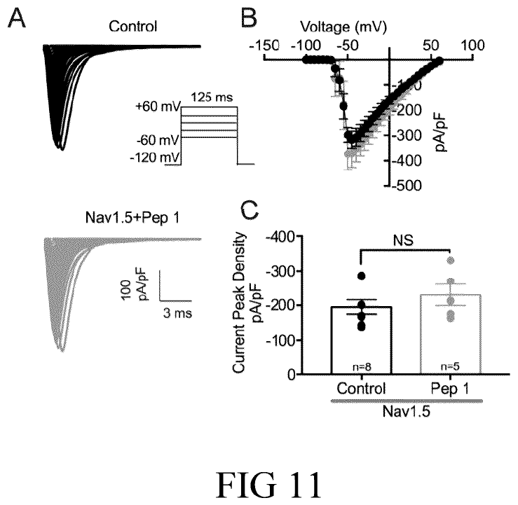

To test whether Peptide 1, 3, 7 and 8 had any modulatory effects on Na_{V} 1.5-mediated Na⁺ currents, we used whole-cell patch-clamp electrophysiology in HEK293 cells stably expressing Na_{V}1.5 (HEK-Na_{V}1.5) channels; each group was either treated with peptides 1, 3, 7 and 8 (500 nM from a stock solution dissolved in H₂O or DMSO) or H₂O and 0.1% DMSO alone (control). The results of these experiments are shown in FIGS. 11-14.

Other embodiments of the invention will be apparent to those skilled in the art from consideration of the specification and practice of the invention disclosed herein, It is intended that the specification and examples be considered as exemplary only, with a true scope and spirit of the invention being indicated by the following claims.

1. A composition comprising of a peptide comprising an amino acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11, or a pharmaceutically acceptable salt, prodrug, or solvate thereof.

2. The composition of claim 1, wherein the peptide inhibits Na_{V}1.7 sodium channel activity.

3. The composition of claim 1, wherein the peptide inhibits Na_{V}1.7 sodium channel activity relative to Na_{V}1.5 sodium channel activity.

4. The composition of claim 1, wherein the peptide inhibits Na_{V}1.7 sodium channel activity relative to Na_{V}1.6 sodium channel activity.

5. The composition of claim 1, wherein the peptide inhibits Na_{V}1.7 sodium channel activity relative to Na_{V}1.4 sodium channel activity.

6. A pharmaceutical composition, comprising the peptide of claim 1 and a pharmaceutically acceptable carrier.

7. A method of treating pain, said method comprising administering an effective amount of the of the composition of claim 1, to a subject in need thereof.

8. The method of claim 7, wherein said pain is neuropathic pain.

9. The method of claim 7, wherein said pain is chronic pain.

10. The method of claim 7, wherein pain is acute pain.

11. The method of claim 7, wherein said pain is inflammatory pain.

12. The method of claim 7, wherein said pain is surgical pain.

13. The method of claim 7, wherein said peptide is administered to said subject by one or more of the oral, buccal, mucosal, sublingual, parenteral, subcutaneous, intramuscular, intraperitoneal, intrathecal, intranasal, inhalation, transdermal, rectal or vaginal routes.

14. A method for manufacturing a medicament for treating pain, which comprises combining the composition of claim 1 and and a pharmaceutically acceptable carrier.

15. A method of preventing pain, comprising administering a prophylactically effective amount of the composition claim 1.

16. The composition of claim 1, wherein the peptide comprises an amino acid sequence selected from SEQ ID NO: 1, SEQ ID NO: 2, SEQ ID NO: 3, SEQ ID NO: 7, SEQ ID NO: 8, SEQ ID NO: 9, SEQ ID NO: 10, and SEQ ID NO: 11, or a pharmaceutically acceptable salt, prodrug, or solvate thereof.

17. The composition of claim 1, wherein the peptide comprises an amino acid sequence selected from SEQ ID NO: 1, SEQ ID NO: 3, and SEQ ID NO: 11, or a pharmaceutically acceptable salt, prodrug, or solvate thereof.

18. The composition of claim 1, wherein the peptide comprises an amino acid sequence selected from SEQ ID NO: 2, SEQ ID NO: 7, and SEQ ID NO: 8, or a pharmaceutically acceptable salt, prodrug, or solvate thereof.

19. The composition of claim 1, wherein the peptide comprises an amino acid sequence selected from SEQ ID NO: 9 and SEQ ID NO: 10, or a pharmaceutically acceptable salt, prodrug, or solvate thereof.

20. A method of treating pain, said method comprising administering an effective amount of the of the composition of claim 19.


SEQUENCE LISTING (11 sequences)

SEQ ID NO: 1 - protein, 16 aa, Artificial Sequence
  Synthetic Peptide 1
SDEIPATFGGGTDAGL

SEQ ID NO: 2 - protein, 34 aa, Artificial Sequence
  Synthetic Peptide 3
DCLGLFRKCIPDMLKCCRFNLVCSRLHKWCKYVF

SEQ ID NO: 3 - protein, 34 aa, Artificial Sequence
  Synthetic Peptide 4
DCLGLFRKCIPDNDKCCRPNFVCSRTHKVCFYVL

SEQ ID NO: 4 - protein, 34 aa, Artificial Sequence
  Synthetic Peptide 5
DCLGLFRKCIPDNDKCCRPNLVCSRTHKVCFYVL

SEQ ID NO: 5 - protein, 34 aa, Artificial Sequence
  Synthetic Peptide 6
DCLGMFRKCLPDDDKCCRPNLVCSRTHKWCRLVL

SEQ ID NO: 6 - protein, 33 aa, Artificial Sequence
  Synthetic Peptide 7
IGEKVTIRLITSTNINDDFNIYQQKPGEPPKLL

SEQ ID NO: 7 - protein, 39 aa, Artificial Sequence
  Synthetic Peptide 8
DCLGFMRKCLSTTDLDDDWNCCRPNLVCSRTHKWCKYVF

SEQ ID NO: 8 - protein, 29 aa, Artificial Sequence
  Synthetic Peptide 10
YCQRFMLTCDSKKACCEGLRCKLLCRKII

SEQ ID NO: 9 - protein, 29 aa, Artificial Sequence
  Synthetic Peptide 11
YCQRWLWTCDSKKACCEGLRCKLWCRKII

SEQ ID NO: 10 - protein, 30 aa, Artificial Sequence
  Synthetic Peptide 12
YCLGFMRKCDSERKCCEGMVCRLWCKRRLW

SEQ ID NO: 11 - protein, 29 aa, Artificial Sequence
  Synethtic Peptide 13
SCRRAWMACDTAKVCCNPIKCRVACKRVL